(19) United States
(12) Patent Application Publication
(10) Pub. No.: US 2014/0083427 A1
(43) Pub. Date: Mar. 27, 2014

(54) PATIENT INTERFACE DEVICE INCLUDING PRESSURE RELIEF FOR DEFORMABLE COMPENENTS

(71) Applicants: Derrick Blake Andrews, Markleton, PA (US); Richard Thomas Haibach, Verona, PA (US); Anthony Vincent Startare, Belle Vernon, PA (US)

(72) Inventors: Derrick Blake Andrews, Markleton, PA (US); Richard Thomas Haibach, Verona, PA (US); Anthony Vincent Startare, Belle Vernon, PA (US)

(73) Assignee: KONINKLIJKE PHILIPS N.V., EINDHOVEN (NL)

(21) Appl. No.: 14/008,720

(22) PCT Filed: Apr. 12, 2012
(86) PCT No.: PCT/IB2012/051797

§ 371 Date: Sep. 30, 2013

Related U.S. Application Data
(60) Provisional application No. 61/476,452, filed on Apr. 18, 2011.

(51) Int. Cl. A61M 16/06

(52) U.S. Cl. CPC A61M 16/06
USPC 128/205.25; 128/206.24

(57) ABSTRACT

A patient interface device includes a sealing cushion structured to communicate a flow of breathing gas within an airway of a patient, a rigid or semi rigid support member coupled to the sealing cushion, the support member including an inner surface, an outer surface and at least one orifice extending therethrough, and a cushion member provided on the inner surface of the support member and structured to engage a portion of the head or face of the patient when the patient interface device is donned by the patient, the cushion member being made of a deformable material and overlapping the orifice such that a portion of the cushion member will flow at least partially through the orifice when pressure is applied to the cushion member.

### CROSS-REFERENCE TO RELATED APPLICATIONS

This patent application claims the priority benefit under 35 U.S.C.§119(e) of U.S. Provisional Application No. 61/476,452 filed on Apr. 18, 2011, the contents of which are herein incorporated by reference.

### BACKGROUND OF THE INVENTION

1. Field of the Invention

The present invention relates to respiratory patient interface devices, and, in particular, to a patient interface device having a mechanism for providing pressure relief for deformable components such as cushion members structured to engage the patients head or face.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Adherence and compliance to therapy, such as CPAP or other pressure support therapies, is growing to be an industry-wide issue. Factors such as comfort and ease of use of a patient interface device can greatly affect a patient's adherence and compliance to therapy. Often, soft, cushiony materials, such as gel materials, are incorporated into patient contacting components of patient interface devices in order to increase patient comfort. For example, U.S. Pat. No. 7,665,464 (incorporated herein by reference), assigned to the assignee of the present invention, describes a respiratory mask that includes a forehead cushion made of a gel material, U.S. Pat. No. 7,870,859 (incorporated herein by reference), also assigned to the assignee of the present invention, describes a respiratory mask that includes a patient sealing surface made of a gel material, and U.S. Pat. No. 7,624,735 (incorporated herein by reference), also assigned to the assignee of the present invention, describes a respiratory mask that includes cheek interfaces that are made of a gel substance.

When a gel or a similar cushiony material is bonded to a more rigid material, such as when a gel cushion is bonded to a frame element of a respiratory mask (often made of an injection molded thermoplastic or silicone), the stiffness of the gel is significantly increased, particularly when a force (e.g., form the patient's face) is applied thereto. This increased stiffness is caused by the constraining of the geometry of the gel component that occurs at the plane where the two materials meet. As can be appreciated, the increased stiffness lessens the comfort providing effect that the gel component was intended to provide.

### SUMMARY OF THE INVENTION

In one embodiment, a patient interface device is provided that includes a sealing cushion structured to communicate a flow of breathing gas within an airway of a patient, a rigid or semi rigid support member coupled to the sealing cushion, the support member including an inner surface, an outer surface and at least one orifice extending therethrough, and a cushion member provided on the inner surface of the support member and structured to engage a portion of the head or face of the patient when the patient interface device is donned by the patient, the cushion member being made of a deformable material and overlapping the orifice such that a portion of the cushion member will flow at least partially through the orifice when pressure is applied to the cushion member.

In another embodiment, a method providing a flow of breathing gas to a patient that includes donning a patient interface device by placing the patient interface device on a head of the patient, wherein the patient interface device includes a sealing cushion, a rigid or semi rigid support member coupled to the sealing cushion, the support member including an inner surface, an outer surface and at least one orifice extending therethrough, and a cushion member provided on the inner surface of the support member and structured to engage a portion of the head or face of the patient when the patient interface device is donned by the patient, the cushion member being made of a deformable material and overlapping the orifice. The method further includes applying pressure to the cushion member with the portion of the patient's head or face and thereby causing a portion of the cushion member to flow at least partially through the orifice, and communicating a flow of breathing gas within an airway of the patient through the sealing cushion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to one exemplary embodiment of the present invention;

FIG. 2A is a cross-sectional view of a portion of a patient interface device forming a part of the system of FIG. 1;

FIG. 2B is a cross-sectional view of a portion of a prior art patient interface device;

FIGS. 3A-3D are top plan views of various embodiment of a frame member and cheek cushion of a patient interface device forming a part of the system of FIG. 1;

FIG. 4 is a schematic diagram of a system adapted to provide a regimen of respiratory therapy to a patient according to another exemplary embodiment of the present invention; and

FIG. 5 is a cross-sectional view of a frame member of a patient interface device forming a part of the system of FIG. 1 according to an alternative embodiment.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

As used herein, the term “viscoelastic material” shall refer to a material that exhibits both viscous and elastic characteristics when undergoing deformation, and as a result exhibits time dependent strain. A viscoelastic material will thus deform under the influence of an applied stress, and when the stress is removed from the material, the material will slowly and not instantaneously recover from some but not all of the deformation. One non-limiting example of a viscoelastic material is a gel substance comprising a viscoelastic polyurethane polymer.

As used herein, the term “elastic material” shall refer to a material that exhibits elastic but not viscous characteristics when undergoing deformation. Elastic materials deform under the influence of an applied stress and return instantaneously to their original state once the stress is removed, thereby recovering from all of the deformation.

As used herein, the term “gel material” shall mean a viscoelastic composition having a measurable durometer within the range of about 5 Shore 000 to about 20 Shore A, as measured by the ASTM D2240-00 testing standard. The composition can be composed of one viscoelastic material, a combination of viscoelastic materials, or a viscoelastic material or materials housed within a bladder or covering, such as a fabric covering.

As used herein, the term “deformable material” shall mean a viscoelastic material or an elastic material.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

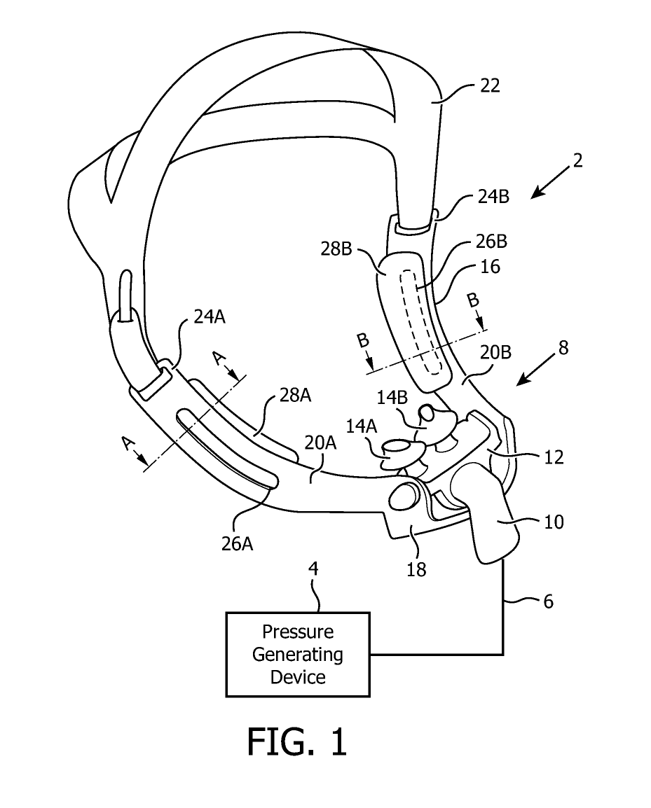

A system 2 adapted to provide a regimen of respiratory therapy to a patient according to one exemplary embodiment is generally shown in FIG. 1. System 2 includes a pressure generating device 4, a delivery conduit 6, and a patient interface device 8 having a fluid coupling conduit 10. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10, which in the illustrated embodiment is an elbow connector. Delivery conduit 6 and patient interface device 8 are often collectively referred to as a patient circuit.

In the illustrated embodiment, patient interface 8 comprises a nasal cushion having nasal prongs that are received within the patient's nares. Any type of patient interface device 8, however, such as a nasal mask that covers the nose, a nasal/oral mask that covers the nose and mouth, nasal saddle type cushion structured to be placed against the lower, underside portion of the nose of a patient (wherein the patient's nares are engaged and covered), or a full face mask that covers the patient's face, which facilitates the delivery of the flow of breathing gas to, and the removal of a flow of exhalation gas from, the airway of such a patient, may be used while remaining within the scope of the present invention. In the embodiment shown in FIG. 1, patient interface 8 includes a cushion 12 having nasal prongs 14A and 14B that is coupled to a frame member 16.

In the illustrated embodiment, cushion 12 is defined from a unitary piece of soft, flexible, cushiony, elastomeric material, such as, without limitation, silicone, an appropriately soft thermoplastic elastomer, a closed cell foam, or any combination of such materials. An opening in cushion 12 to which fluid coupling conduit 10 is coupled allows the flow of breathing gas from pressure generating device 4 to be communicated to an interior space defined by cushion 12, and then to the airway of a patient. The opening in cushion 14 also allows the flow of exhalation gas (from the airway the patient) to be communicated to an exhaust port that may be provided, for example and without limitation, in fluid coupling conduit 10 or elsewhere on the patient interface device 8.

In the illustrated embodiment, frame member 16 is made of a rigid or semi-rigid material, such as, without limitation, an injection molded thermoplastic or silicone, and includes a cushion attachment portion 18 to which cushion 12 is attached. Frame member 16 also includes first and second arms 20A, 20B extending from opposites sides of cushion attachment portion 18. First and second arms 20A, 20B are structured to extend over the patient's cheeks when patient interface 8 is donned by the patient.

As seen in FIG. 1, the patient interface 8 further includes a headgear assembly 22 that is adjustably coupled to looped connecting members 24A, 24B provided at the distal ends of arms 20A, 20B, respectively. Headgear assembly 22 is configured to secure patient interface device 8 to the patient's head in a manner wherein cushion 14 is held in place beneath the patient's nose with nasal prongs 14A, 14B being partially received within the patient's nares. This enables gasses to be communicated to and from the airway of the patient through cushion 12.

As shown in FIG. 1, each arm 20A, 20B includes a window or orifice 26A, 26B that extends entirely through the arm 20A, 20B. In the illustrated embodiment, each orifice 26A, 26B has an oblong shape which extends along a portion of (e.g., one-third to one-half of) the associated arm 20A, 20B. In addition, a cheek cushion 28A is provided on the interior surface of arm 20A, and a cheek cushion 28B is provided on the interior surface of arm 20B. In particular, each cheek cushion 28A, 28B is positioned on the interior surface of the associated arm 20A, 20B in a configuration in which it overlaps the associated orifice 26A, 26B.

Cheek cushions 28A, 28B are structured to engage the cheeks of the patient when patient interface device 8 is donned by the patient. Thus, to provide comfort to the patient, cheek cushions 28A, 28B are made of a cushiony, deformable material. In the exemplary, non-limiting embodiment, cheek cushions 28A, 28B are each made of a gel material and are overmolded on the arms 20A, 20B using, for example, an insert molding or two shot injection molding technique. More specifically, the harder arms 20A, 20B would be first molded with the orifices 26A, 26B extending therethrough, and then the softer cheek cushions 28A, 28B would be molded to overlap the associated orifice 26A, 26B. Cheek cushions 28A, 28B may also be made of other deformable materials, such as, without limitation, silicone, silicone gel, polyurethane gel, closed cell foam, open cell foam, TPE (thermoplastic elastomer), or any one of these materials within a bladder or covering.

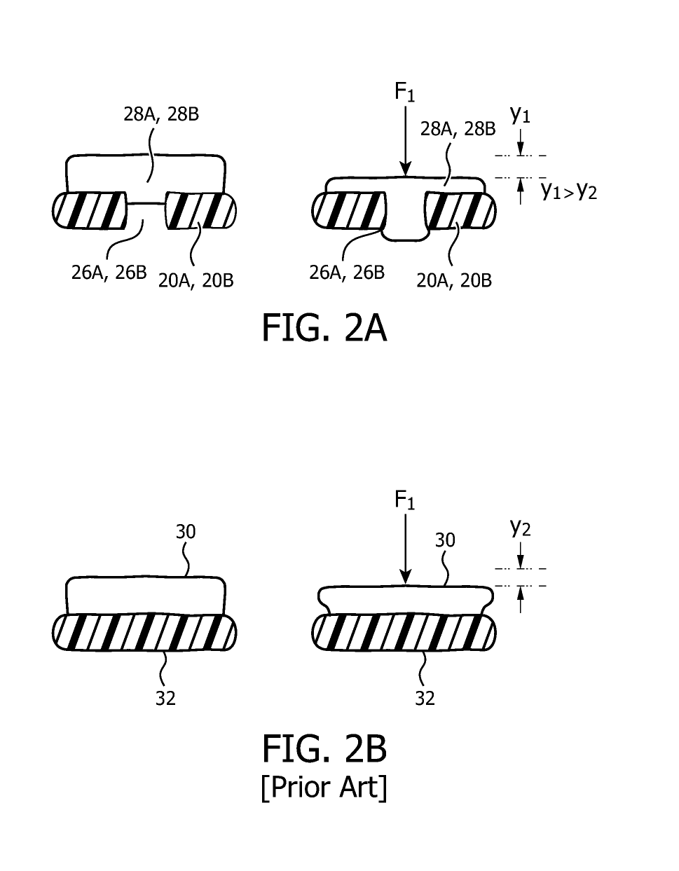

The advantageous properties provided by the combination of cheek cushions 28A, 28B and orifices 26A, 26B will now be described in connection with FIGS. 2A and 2B. In particular, as shown in FIG. 2A, which are cross-sectional views taken along lines A-A or B-B in FIG. 1, the orifices 26A, 26B allow the deformable (e.g., gel) material of cheek cushions 28A, 28B to partially flow through the arms 20A, 20B (through the orifices 26A, 26B) when a generally perpendicular force F₁ is applied to a top surface of the cheek cushion 28A, 28B (such as when the patient interface device 8 is donned by the patient). This, in turn, reduces the stiffness/increases the softness (both actual and perceived) of the cheek cushions 28A, 28B. This is in contrast to prior art cheek cushions 30 provided on prior art frame members 32 as shown in FIG. 2B, which experience increased stiffness in response to similar forces. In addition, as illustrated in FIGS. 2A and 2B, cheek cushions 28A, 28B are able to be vertically displaced and deformed to a larger degree than prior art cheek cushions 30 in response to similar perpendicular forces F₁ (see y₁>y₂ in FIGS. 2A and 2B).

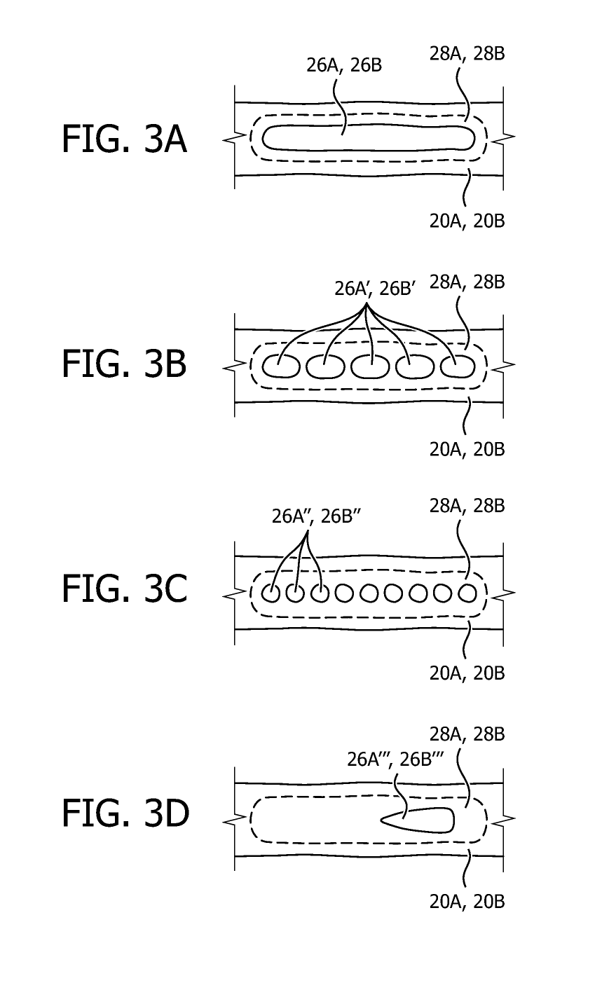

As noted elsewhere herein, orifices 26A, 26B can take on any of a number of different shapes, and may be provided in different numbers. In the illustrated embodiment, orifices 26A, 26B are each a single orifice and each have an oblong shape as shown in FIG. 1 and in FIG. 3A. FIGS. 3B-3D illustrate a number of alternative orifice configurations. In particular, FIG. 3B shows a plurality of smaller oblong orifices 26A′, 26B′ provided in arms 20A, 20B, FIG. 3C shows a plurality of circular orifices 26A″, 26B″ provided in arms 20A, 20B, and FIG. 3D shows a single, three sided (generally triangular) orifice 26A″′ provided in arms 20A, 20B. As seen in FIG. 3D, orifice 26A″′ is provided in a particular, limited region of arms 20A, 20B in order to provide localized softness control for a particular area of cheek cushions 28A, 28B (and thus a particular area of the patient's face or head (e.g., the cheekbones)) without having to change the overall durometer of the cheek cushions 28A, 28B. As will be appreciated, still other shapes and numbers of orifices are possible.

In one particular, non-limiting embodiment, orifices 26A, 26B (with rounded ends) are 4 mm wide, 55 mm long, and are provided in arms 20A, 20B made of plastic that are 2.5 mm thick. In addition, in this embodiment, cheek cushions 28A, 28B are 13 mm wide, 65 mm long, and 3.5 mm thick.

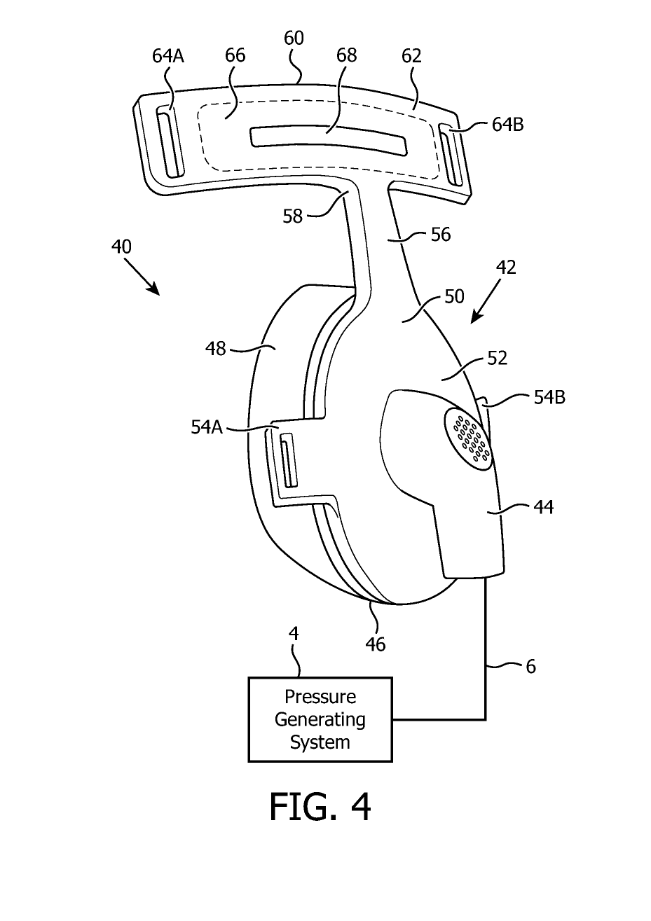

A system 40 adapted to provide a regimen of respiratory therapy to a patient according to another exemplary embodiment is generally shown in FIG. 4. System 40 includes a number of the same components as system 2 of FIG. 1, and like components are labeled with like reference numerals. As seen in FIG. 4, system 40 includes a pressure generating device 4 and a delivery conduit 6 as described elsewhere herein, and a patient interface device 42 having a fluid coupling conduit 44, which in the illustrated embodiment is an elbow connector.

Patient interface device 40 includes a patient sealing assembly 46, which in the illustrated embodiment is a nasal/oral mask. However, other types of patient sealing assemblies, such as, without limitation, a nasal mask or a full face mask, which facilitates the delivery of the flow of breathing gas to the airway of a patient, may be substituted for patient sealing assembly 46 while remaining within the scope of the present invention. Patient sealing assembly 46 includes a cushion 48 coupled to a frame member 50. In the illustrated embodiment, cushion 48 is defined from a unitary piece of soft, flexible, cushiony, elastomeric material, such as, without limitation, silicone, an appropriately soft thermoplastic elastomer, a closed cell foam, a gel or any combination of such materials. Also in the illustrated embodiment, frame member 50 is made of a rigid or semi-rigid material, such as, without limitation, an injection molded thermoplastic or silicone, and includes a faceplate portion 52 to which cushion 48 is fluidly attached.

An opening in faceplate portion 52, to which fluid coupling conduit 44 is coupled, allows the flow of breathing gas from pressure generating device 4 to be communicated to an interior space defined by cushion 48, and then to the airway of a patient. In an alternative embodiment, cushion 48 may be supported by and received through an orifice provided in frame member 50 so that fluid coupling conduit 44 can be directly connected to cushion 48 rather than to a faceplate portion. In addition, in the exemplary embodiment, faceplate portion 52 includes first and second connecting members 54A, 54B for receiving a respective strap of a headgear component (not shown) to secure patient interface device 42 to the patient's head.

Frame member 50 also includes an elongated connecting member 56 having a distal end 58 that is connected to an forehead cushion assembly 60 of patient interface device 42. Forehead cushion assembly 60 includes forehead cushion support member 62 that is made of a rigid or semi-rigid material, such as, without limitation, an injection molded thermoplastic or silicone, and includes first and second loop connectors 64A, 64B structured to receive a respective upper strap of a headgear component (not shown) to help secure patient interface device 42 to the patient's head. A forehead cushion 66, shown in phantom lines, is coupled to the rear side of forehead cushion support member 62.

Forehead cushion 66 is made of a cushiony, deformable material, which in the exemplary, non-limiting embodiment is a gel material as described herein. In addition, an oblong orifice 68 is provided though forehead cushion support member 62. Forehead cushion 66, which is structured to engage the patient' forehead, is positioned on the rear surface of the forehead cushion support member 62 in a configuration in which it overlaps orifice 68. As described elsewhere herein, the orifice 68 allows the deformable (e.g., gel) material of forehead cushion 66 to partially flow through forehead cushion support member 62 (through orifices 66) when a generally perpendicular force is applied to a top surface of the forehead cushion 66 (such as when the patient interface device 42 is donned by the patient). This in turn reduces the stiffness/increases the softness (both actual and perceived) of the forehead cushion 66.

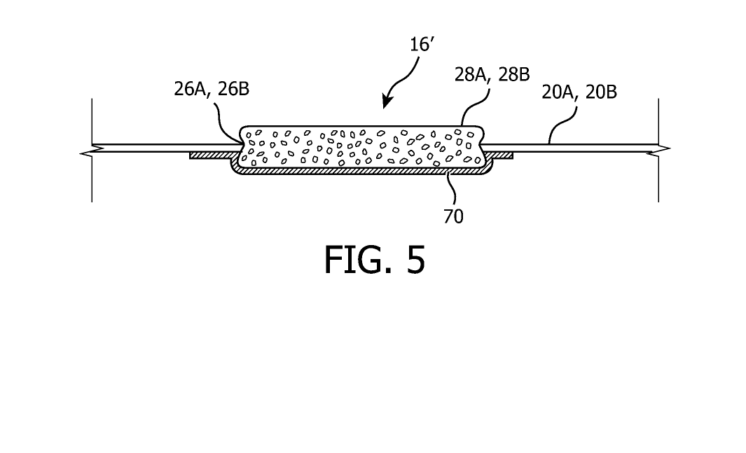

FIG. 5 is a cross-sectional view of a frame member 16′ according to an alternative embodiment. Frame member 16′ includes arm 20A, 20B having orifice 26A, 26B provided therein as described elsewhere herein. Frame member 16′ also includes cheek cushion 28A, 28B (made of a deformable material as described herein) provided on the interior surface of arm 20A, 20B in a manner that overlaps orifice 26A, 26B. In addition, frame member 16′ includes a covering layer 70, made of, for example and without limitation, a fabric material or a thin film material, that covers the top (face engaging) surface of cheek cushion 28A, 28B. In the illustrated embodiment, covering layer 70 also covers a portion of the interior surface of arm 20A, 20B. The bottom surface of cheek cushion 28A, 28B is not covered by covering layer 70, so that it is able to flow through orifice 26A, 26B as described elsewhere herein when a force is applied to the top surface of cheek cushion 28A, 28B. A similar covering layer 70 may also be provided on top (forehead engaging) surface of the forehead cushion 66 of the system 40 shown in FIG. 4.

Thus, in the various embodiments described herein, the patient interface device includes a feature/mechanism which provides increased comfort to the patient that, as a result, should positively affect a patient's adherence and compliance to therapy.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a sealing cushion structured to communicate a flow of breathing gas within an airway of a patient;
a rigid or semi rigid member having an attachment portion and an arm extending from the attachment portion, wherein the sealing cushion is attached to the attachment portion, the arm including an inner surface, an outer surface and at least one orifice extending therethrough; and
a cheek cushion provided on the inner surface of the arm and structured to engage a check of the patient when the patient interface device is donned by the patient, the cheek cushion being made of a deformable material and overlapping the orifice such that a portion of the cheek cushion will flow at least partially through the orifice when pressure is applied to the cushion member.

2. (canceled)

3. The patient interface device according to claim 1, wherein the deformable material is a gel material.

4. The patient interface device according to claim 1, wherein the at least one orifice comprises a plurality of orifices provided along a length of the arm, and wherein the cheek cushion overlaps each of the orifices.

5. The patient interface device according to claim 4, wherein each of the orifices is a circular orifice.

6. The patient interface device according to claim 4, wherein each of the orifices is an oblong orifice.

7. The patient interface device according to claim 1, wherein the at least one orifice is a three sided, generally triangular orifice.

8. The patient interface device according to claim 1, further comprising a covering layer provided on top of an inner surface of the cheek cushion and on top of a portion of the inner surface of the arm, wherein the cheek cushion engages the cheek of the patient through the covering layer.

9. A method providing a flow of breathing gas to a patient, comprising:
(a) donning a patient interface device by placing the patient interface device on a head of the patient, the patient interface device including:
(1) a sealing cushion,
(2) a rigid or semi rigid frame member having an attachment portion and an arm extending from the attachment portion, wherein the sealing cushion is attached to the attachment portion, the arm including an inner surface, an outer surface and at least one orifice extending therethrough, and
(3) a cheek cushion provided on the inner surface of the arm and structured to engage a cheek of the patient when the patient interface device is donned by the patient, the cheek cushion being made of a deformable material and overlapping the orifice;
(b) applying a pressure to the cheek cushion with the cheek and thereby causing a portion of the cheek cushion to flow at least partially through the orifice; and
(c) communicating the flow of breathing gas within an airway of the patient through the sealing cushion.

10. (canceled)

11. The method according to claim 9, wherein the deformable material a gel material.

12. The method according to claim 9, wherein the at least one orifice comprises a plurality of orifices provided along a length of the arm, and wherein the cheek cushion overlaps each of the orifices.

13. The method according to claim 12, wherein each of the orifices is a circular orifice.

14. The method according to claim 12, wherein each of the orifices is a oblong orifice.

15. The method according to claim 9, wherein the at least one orifice is a three sided, generally triangular orifice.

16. The patient interface device according to claim 1, further comprising a covering layer provided on a top surface of the cheek cushion but not on a bottom surface of the cheek cushion opposite the top surface, the top surface facing away from the inner surface of the arm such that the cheek cushion is structured to engage the cheek of the patient through the covering layer.

17. The method according to claim 9, wherein a covering layer is provided on a top surface of the cheek cushion but not on a bottom surface of the cheek cushion opposite the top surface, the top surface facing away from the inner surface of the arm such that the cheek cushion is structured to engage the cheek of the patient through the covering layer.
